(19) United States
(12) Plant Patent Application Publication
(10) Pub. No.: US 2007/0174941 P1
(43) Pub. Date: Jul. 26, 2007

(54) Alstroemeria plant named 'Mauve Majesty'

(76) Inventor: Mark P. Bridgen, Southold, NY (US)

Correspondence Address: JONDLE & ASSOCIATES P.C., 858 HAPPY CANYON ROAD SUITE 230, CASTLE ROCK, CO, 80108, US

(21) Appl. No.: 11/339,765

(22) Filed: Jan. 25, 2006

(51) Int. Cl. A01H 5/00

(52) U.S. Cl. PLT/309

(57) ABSTRACT

An Alstroemeria cultivar particularly distinguished by its mauve flower color, its strong, upright flower stems, its winter hardiness, and its continuous flowering from beginning of summer until the first hard freeze in the fall is disclosed.

### GENUS AND SPECIES

Alstroemeria hybrid L.

### VARIETY DENOMINATION

‘Mauve Majesty’

### BACKGROUND OF THE NEW PLANT

The present invention comprises a new and distinct cultivar of Alstroemeria, botanically known as Alstroemeria hybrid L., and hereinafter referred to by the cultivar name ‘Mauve Majesty’. ‘Mauve Majesty’ originated from a hybridization made in 2002 in Riverhead, N.Y. The female parent was an Alstroemeria aurea plant (unpatented), while the male parent was the proprietary Alstroemeria plant ‘Purple 1’ (unpatented).

Fifteen days after pollination, embryos were aseptically removed in ovulo and cultured on a one-quarter strength Murashige and Skoog medium. When the embryos germinated in vitro, they were subcultured to full-strength Murashige and Skoog medium for continued development. Once they were large enough to remove from the culture vessel, they were acclimated and grown in the greenhouse for trial. A single plant selection was chosen for further evaluation and for asexual propagation in 2002 in Riverhead, N.Y.

‘Mauve Majesty’ was selected for its distinctive mauve flower color, continuous flowering, and strong, upright flower stems. It was also selected because it is winter hardy to temperatures as low as those experienced in USDA zone 5. ‘Mauve Majesty’ has been asexually propagated repeatedly by rhizome division and tissue culture since 2002 and has been found to retain its distinctive characteristics through successive asexual propagations.

### DESCRIPTION OF PHOTOGRAPHS

This new Alstroemeria plant is illustrated by the accompanying photographs which show blooms and foliage of the plant; the colors shown are as true as can be reasonably obtained by conventional photographic procedures.

FIG. 1 shows the overall plant habit.

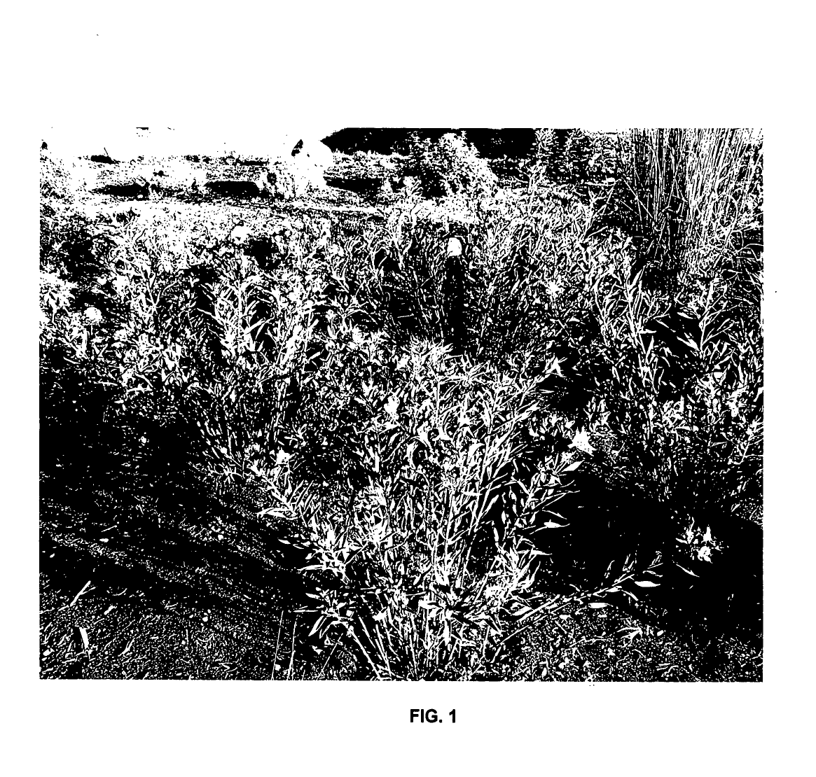

FIG. 2 shows mature inflorescences, flower buds, and leaves.

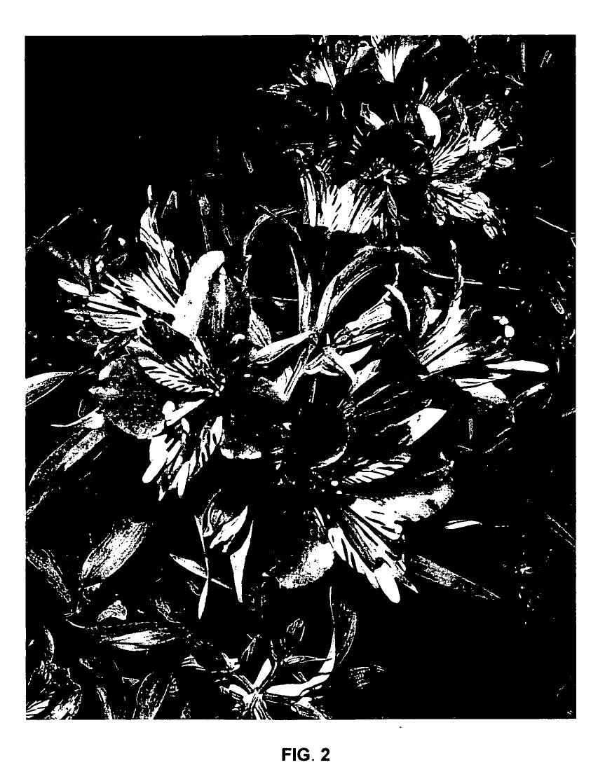

### DESCRIPTION OF THE NEW CULTIVAR

The following detailed descriptions set forth the distinctive characteristics of ‘Mauve Majesty’. The data which define these characteristics were collected from asexual reproductions carried out in Riverhead, N.Y. The data were collected on plants grown under field conditions in full sun. Color references are primarily to the RHS Colour Chart of The Royal Horticultural Society of London (RHS).

### DETAILED BOTANICAL DESCRIPTION

Classification:
   Botanical.—Alstroemeria hybrid L.
   Common name.—Alstroemeria, Lily-of-the-Incas; Inca Lily; Peruvian Lily.
Parentage:
   Female parent.—Alstroemeria aurea (unpatented).
   Male parent.—Proprietary Alstroemeria plant ‘Purple 1’ (unpatented).
Plant:
   Origin.—Seedling derived from ovule-cultured hybrid embryos.
   Form.—Herbaceous plant arising from an underground rhizome.
   Habit.—Upright and vigorous.
   Plant size.—Height: 75-80 cm — Width (Spread): 90-100 cm.
   Flowering habit.—Continuous from the beginning of summer until the first hard freeze in fall.
   Time to bloom from propagation.—If a clump is divided in the spring or early summer, it will resume flowering within a month.
Flowering stems:
   Color.—RHS 137A or RHS 137C.
   Description.—Long, strong, and upright growing floral stems.
   Size: Length: 62.5-80 cm — Diameter: 1-2 cm.
   Internode length.—Not applicable.
   Texture.—Glabrous leaf surface.
   Color.—RHS 137A (Dark green) on both upper and under sides.
   Vegetative stem length.—70-75 cm.
Leaves:
   Arrangement.—Simple, linear, resupinate leaves with parallel venation that are arranged alternately.
   Shape.—Elongated elliptic with extended bases.
   Apex.—Broadly acuminate apices.
   Base.—Extended.
   Margin.—Entire.
   Color.—Upper surface: RHS 137A or RHS 137C — Lower surface: RHS 137A or RHS 137C.
   Size.—Length: 10 cm — Width: 2.2 cm.
   Texture.—Glabrous leaf surface.
   Venation.—Parallel.
   Pubescence.—None.
   Petiole.—None (sessile leaves).
Flowers:
   Inflorescence type.—A terminal bracted umbel of cymes.
   Flowering season.—Summer through early fall.
   Lastingness of individual flowers on the plant.—15-21 days.
   Color.—RHS 70B to RHS 71B.
   Flower width.—Approximately 5 cm at the open end
   Flower depth.—3.7-4.2 cm.
   Flower shape.—Tubular.
   Fragrance.—None.
   Number of flowers per plant.—Everblooming; varies with size of plant; 30-50 flowers per plant over 3 months.
   Number of florets per umbel.—Typical: 15 — Range: 10-20.
Peduncle:
   Length.—2.5-4 cm.
   Diameter.—Approximately 0.9 -1 cm before opening.
Flower buds:
   Color.—RHS 70B to RHS 71B.
   Shape.—Pear-shaped becoming long and more pointed just before opening.
   Size.—Length: Approximately 2.5 to 2.8 cm before opening — Diameter: Approximately 0.9 to 1 cm before opening.
Tepals:
   Arrangement.—Two concentric circles of three tepals each.
   Shape.—Tubular/spatulate to oblanceolate-spatulate.
   Size: Length: 3.7-4.2 cm — Width: Approximately 5 cm at the tip.
   Apex.—Emarginate to mucronate to apiculate apices.
   Texture.—Glabrous.
   Color.—RHS 71B on young flowers fading slightly to RHS 70B on older flowers.
Reproductive organs:
   Pistils.—Number: 1 — Style length: 2.5 -4.0 cm.
   Stamens.—Number: 6.
   Filaments.—Length: Approximately 2.5-4.0 cm.
   Pollen.—Flowers are sterile, therefore no pollen is produced.
   Seeds.—None.
Disease and insect resistance: Because this plant is sterile, there is no pollen and thrips are less likely to attack.

### COMPARISON WITH PARENTAL AND COMMERCIAL CULTIVARS

The female parent, Alstroemeria aurea (unpatented), is a species that is native to southern Chile and it differs from ‘Mauve Majesty’ by having yellow to orange flowers and by flowering only in the spring and early summer whereas ‘Mauve Majesty’ has mauve flowers and flowers continuously from the beginning of summer until the first hard freeze in fall.

The male parent, ‘Purple 1’ (unpatented), has larger flowers than ‘Mauve Majesty’ with a different shade of purple and is not as floriferous as ‘Mauve Majesty’. In addition, ‘Purple 1’ is not hardy to USDA zone 5, whereas ‘Mauve Majesty’ is hardy to USDA zone 5.

‘Mauve Majesty’ differs from the commercial Alstroemeria cultivar ‘Sweet Laura’ (U.S. Plant Pat. No. 10,030), by having mauve flowers that lack fragrance, whereas ‘Sweet Laura’ has yellow flowers that are fragrant. Both cultivars are winter hardy to USDA zone 5.

I claim:

1. A new and distinct cultivar of Alstroemeria plant as described and shown herein.
